(19) United States
(12) Patent Application Publication
(10) Pub. No.: US 2006/0115379 A1
(43) Pub. Date: Jun. 1, 2006

(54) Magnetostrictive pump

(76) Inventors: David T. Bach, Ellicott City, MD (US); Muniswamappa Anjanappa, Ellicott City, MD (US)

Correspondence Address: ROYAL W. CRAIG, SUITE 153, 10 NORTH CALVERT STREET, BALTIMORE, MD, 21202, US

(21) Appl. No.: 11/273,583

(22) Filed: Nov. 14, 2005

Related U.S. Application Data
(63) Continuation-in-part of application No. 10/688,331, filed on Oct. 17, 2003.
(60) Provisional application No. 60/627,383, filed on Nov. 12, 2004.

(51) Int. Cl. G01N 33/00

(52) U.S. Cl. 422/73; 417/321

(57) ABSTRACT

A multi-valved positive displacement pump design that employs a magnetostrictive fluid pump in combination with magnetostrictive gates. The pump generally comprises a pump housing defining an internal fluid chamber, an inlet port to the fluid chamber, an outlet port from the fluid chamber, a magnetostrictive positive displacement pump in the fluid chamber, and magnetostrictive gates (valves) selectably blocking the inlet port to the fluid chamber and the outlet port from the fluid chamber. All pump and gate actuators include rods formed of magnetostrictive material (seated in the housing), and coil windings for electrical excitation to vary the length of the rods. The pump and valves are operated under PLC control according to a pumping sequence comprising a fluid induction step wherein the pump and inlet actuator rods are retracted and the outlet actuator rod is extended to open the inlet port and induct fluid into the fluid chamber, and a fluid dispensing step wherein the pump and inlet actuator rods are extended and the outlet actuator rod is retracted to close the inlet port and expel fluid from the fluid chamber. The positive displacement pump has particular utility in the context of a cell sorter system for sorting desired cells from undesired matter, and an embodiment thereof is also disclosed.

### CROSS-REFERENCE TO RELATED APPLICATIONS

The present application derives priority from U.S. Provisional Patent Application No. 60/627,383; filed: 12 Nov. 2004, and is a continuation-in-part of U.S. patent application Ser. No. 10/688,331, filed 17 Oct. 2003.

### BACKGROUND OF THE INVENTION

1. Field of the Invention

The present invention relates to pumps and, in more particularly, to a magnetostrictive pump that relies on the expansion and contraction of a primary magnetostrictive rod for volumetric displacement of a pumping chamber, and additional magnetostrictive elements for gating of inlet and outlet valves.

2. Description of the Background

Conventional positive displacement pumps pump liquids in and out of a pumping chamber by changing the volume of the chamber. Typically these pumps are bulky, require a source of mechanical power such as a motor or engine, and have numerous moving parts such as mechanical linkages, gearboxes, etc.

Magnetostrictive fluid pumps are quite different. Rather than a conventional piston, they rely on expanding rods that serve as actuators. The rods are made of magnetostrictive material that changes dimensions in the presence of a magnetic field. Thus, the rods move in and out of a pumping chamber like a solenoid, thereby changing the volume of the chamber. The rods may be moved within a range of several tens of microns. There are no moving parts at all, and so magnetostrictive pumps can run reliably over a long period of time.

Numerous magnetostrictive materials are known. For example, European Patent Application No. 923009280 discloses many such materials. A commercially available magnetostrictive material is Terfenol-D® by Etrema Corporation, of Ames, Iowa.

Examples of magnetostrictive pumps include U.S. Pat. No. 5,641,270, which uses a magnetostrictive element to compress a single pumping chamber. The rod produces a single pumping compression stroke for each cycle of contraction and expansion of the magnetostrictive material. Single-stroke pumps are inherently prone to pressure fluctuations. In many applications such as laboratory settings, constant pressures and flow rates are critical.

United States Patent Application 20050147506 by Dooley published Jul. 7, 2005 shows a multi pumping chamber magnetostrictive pump that facilitates higher flow rates, and smoother fluid delivery. A single magnetorestrictive actuator drives multiple pumping chambers, such as two inline pumping chambers by the linear expansion of the actuator at both ends. A pump assembly having multiple pumps each including a magnetostrictive element is also disclosed.

While the Dooley application suggests multi-chambered pumps, it fails to suggest how multiple magnetostrictive elements can be used for precision dispensing applications in a clean room environment. This requires a valved-intake port for fluid or air induction, and a valved-output port for dispensing into container, syringes or vials, etc.

For example, there is a large demand for cost effective cell sorting of stem and other cell types. Sorted isolated cell populations are used for transplantation into myeloablated cancer patients. There are currently about 100,000 such transplantations a year in the US. Cell sorting ideally needs to take place in a closed consumable container where hematopoeitic stem and progenitor cell populations are sorted from peripheral blood, umbilical cord blood, and bone marrow. Automated cell sorting techniques are becoming indispensable for both research and clinical applications. However, current cell sorters require constant parameter adjustments, use open flow sorting technology which is susceptible to contamination and these sorters can cost in excess of $250,000. These devices detect the properties of cells, and implement the physical separation of cells of interest at high speed. The detection of cells are done using optical techniques such as fluorescence and light scattering followed by separation of the cells of interest using electrostatic or other physical separation methods. Conventional cell sorters utilize a single channel that operates at sorter rates of up to 60,000 cells per second. The sorting is done using an open fluid flow system that creates an aerosol environment. This open fluid flow environment creates a high potential for contamination. A magnetostrictive pump can close the flow environment, resulting in an isolated fluid system.

It would be greatly advantageous to provide a positive displacement pump design that employs a magnetostrictive fluid pump in combination with magnetostrictive gates for high-throughput and high-accuracy filling and dispensing applications, with particular utility as a cell sorter having a closed disposable consumable portion that removes the risk of contamination.

### SUMMARY OF THE INVENTION

Accordingly, it is an object of the present invention to provide a positive displacement pump design that employs a magnetostrictive fluid pump in combination with magnetostrictive gates for high-throughput and high-accuracy filling and dispensing applications.

It is another object to provide a positive displacement pump design for high-accuracy filling and dispensing applications that can be micromachined, creating a disposable pump consumable.

It is another object to provide a positive displacement pump design as described above that has particular utility as a cell sorter.

According to the present invention, the above-described and other objects are accomplished by providing a positive displacement pump design that employs a magnetostrictive fluid pump in combination with magnetostrictive gates. The pump generally comprises a pump housing defining an internal fluid chamber, an inlet port to the fluid chamber, an outlet port from the fluid chamber, a magnetostrictive positive displacement piston in the fluid chamber, and magnetostrictive gates (valves) selectably blocking the inlet port to the fluid chamber and the outlet port from the fluid chamber. All pump and gate actuators include rods formed of magnetostrictive material with a stainless steel, ceramic or polymer tip that is seated in the housing that comes in contact with the fluid, and coil windings for electrical excitation to produce a magnetic field that varies the length of the rods. The pump is operated under electric control according to a pumping sequence comprising a fluid induction step wherein the positive displacement piston is actuated to retract within the pump housing, thereby inducting fluid. The outlet actuator rod remains in its normally extended position that closes the outlet port. Once the positive displacement mechanism is fully retracted and fluid has filled the chamber, the inlet valve is actuated to be closed, the outlet valve is actuated to be open and the positive displacement mechanism is deactivated, allowing the magnetostrictive rod material to expand and return the piston to its normally-extended position, thereby pushing a pulse of fluid through the outlet port of the pump. After moving the pulse of fluid out of the pump housing the outlet valve is deactivated to close the outlet valve, and the inlet valve is deactivated to open the inlet valve. The cycle may repeat.

The positive displacement pump is shown in the context of a cell sorter system for sorting desired cells from undesired matter. The cell sorter includes a precision magnetostrictive pump for pumping cell-containing fluid into a capillary detection and gating region and controlling positions of said cells in said detection/gating region, an optical detection system for measuring a cell characteristic, and a pair of capillary outlets each controlled by a magnetostrictive gate that causes a desired cell to pass through a cell selection port or a normal position port for unwanted material. The select gate, pump and detection system can be controlled and synchronized by a microprocessor. Optical detection can be based on fluorescence, scattered light or both. Other detection techniques such as absorbance are also included in this application.

### BRIEF DESCRIPTION OF THE DRAWINGS

Other objects, features, and advantages of the present invention will become more apparent from the following detailed description of the preferred embodiment and certain modifications thereof when taken together with the accompanying drawings in which:

FIG. 1 shows a preferred embodiment of the magnetostrictive pump, generally comprising a magnetostrictive positive displacement mechanism 2 and magnetostrictive capillary gating mechanism 4 according to the present invention.

FIG. 2 illustrates the pump of FIG. 1 adapted to work as a cell sorter gating system.

FIG. 3 shows the configuration of a magnetostrictive actuator with disposable tip and sensing coil.

FIG. 4 shows an embodiment of a multichannel optical detection system that is suitable for use with the cell sorter embodiment of the present invention as described above.

FIG. 5 is an illustration of a disposable multiple layer micro-fluidic chip version of the cell sorter according to the invention.

FIG. 6 is an isometric view of a disposable microfluidics chip, illustrative of the sequence of operation (A-C) including intake stroke and dispense stroke

FIG. 7 is an overall perspective view of the disposable cell sorter micro-fluidic chip schematicized in FIG. 6

### DETAILED DESCRIPTION OF THE PREFERRED EMBODIMENTS

The present invention is a multi-magnetostrictive pump that employs a magnetostrictive positive displacement element in combination with multiple magnetostrictive valves for high-throughput and high-accuracy filling and dispensing applications. The multi-valved magnetostrictive pump has particular utility in the context of a cell sorter (and an embodiment as such is disclosed), though one skilled in the art should understand that the pump may find application in many other high speed dispensing applications. The pump employs a magnetostrictive gating mechanism comprising magnetostrictive rods coupled to flow ports for selection thereof.

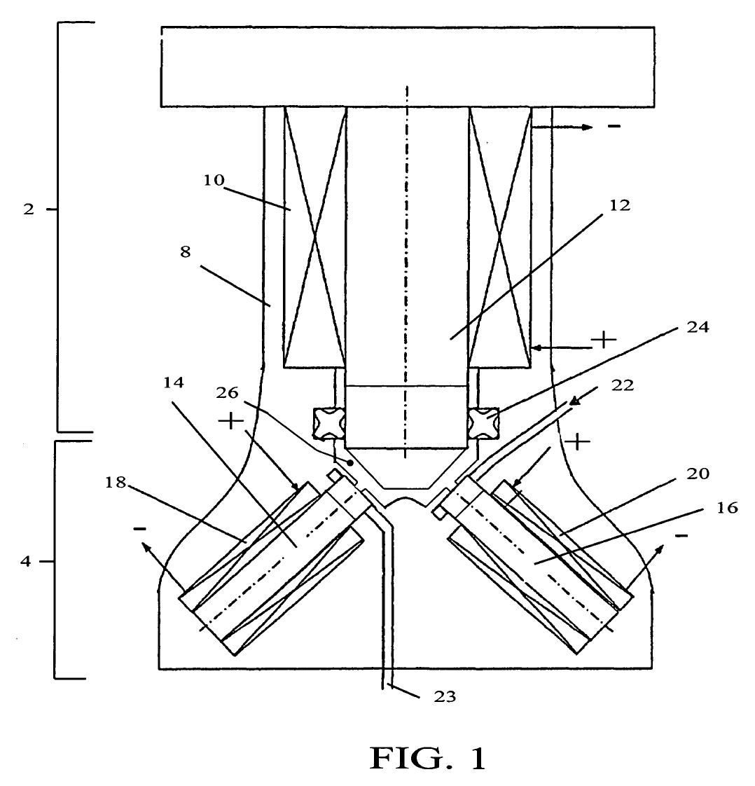

FIG. 1 shows a preferred embodiment of the magnetostrictive pump, generally comprising a magnetostrictive pumping mechanism 2 and magnetostrictive capillary valve mechanism 4 according to the present invention.

The positive displacement mechanism 2 generally comprises a piston actuator rod 12 formed of magnetostrictive material with a permanent magnet bias, and a stainless steel, plastic or ceramic tip that is seated in a pump housing 8. The piston actuator rod 12 extends into a fluid chamber 26, and is sealed therefrom by a distal seal 24 which may be an O-ring seal. Alternately, the sealing system can be a sealless system such as described in SP&S U.S. Pat. No. 6,739,478. The magnetostrictive piston actuator rod 12 is actuated by a coil winding 10 for positive displacement pumping between a normally-extended (unexcited) home position and an extended position. The magnetostrictive gating mechanism 4 is derived from the same fluid chamber 26, and generally comprises a capillary inlet port 22 gated by an inlet valve, and a capillary outlet port 23 gated by an outlet valve. The inlet valve comprises an inlet valve actuator rod 16 formed of magnetostrictive material with either a stainless steel, plastic or ceramic tip, seated in pump housing 8. The inlet valve actuator rod 16 is actuated by coil winding 20 between a normally-retracted (unexcited) open position and an extended closed position, and is in fluid communication with capillary inlet port 22. The outlet valve likewise comprises an outlet valve actuator rod 14 formed of magnetostrictive material and seated in pump housing 8. The outlet valve actuator rod 14 is actuated by coil winding 18 between a normally-extended (unexcited) closed position and an retracted open position, and is in fluid communication with capillary outlet port 23.

In general operation, a voltage is applied to coil winding 10, thereby actuating magnetostrictive piston actuator rod 12 to retract. The magnetostrictive piston actuator rod 12 is excited with a magnetic field that originates outside the fluid chamber 26 and capillary port region. There is no physical contact between the magnetic field generation means and the flow region. The magnetostrictive piston actuator rod 12 and tip, by virtue of its magnetostrictive properties and permanent magnet bias, physically retracts when excited by the externally applied magnetic field induced by coil winding 10, thereby inducting fluid through capillary inlet channel 22. Once the magnetostrictive piston actuator rod 12 is fully retracted and fluid has filled the chamber 26, the inlet valve is actuated to be closed, the outlet valve is actuated to be open, and the positive displacement pump 2 is deactivated, pushing a pulse of fluid through the outlet port of the pump. The outlet valve actuator rod 14 is deactivated and returns to its normally-closed position, and the inlet valve is deactivated to open the inlet valve. The cycle may repeat.

The pump housing 8 defines a gated fluid chamber 26 driven at one end by piston actuator rod 12 to operate as a positive displacement pump, the fluid chamber 26 having a capillary inlet port 22 leading therein, and a capillary outlet port 23 leading therefrom. The ported fluid chamber 26 may be formed as a cylinder having internal dimensions to provide a smooth inflow and outflow of the fluid during the actuation periods. The piston actuator rod 12 enters the fluid chamber 26 from its own chamber, and is distally sealed by the O-ring seal 24 encircling rod 12 at the lower end. The O-ring seal may be made of rubber, Teflon, or other suitable material to assure a proper seal against the operating pressures.

The two valve actuator rods 14, 16, sealed to the housing as in rod 12, are used to gate the capillary inlet port 22 and capillary outlet port 23. The inlet valve actuator rod 16 is actuated by coil winding 20, and is in fluid communication with a capillary inlet port 23. The outlet valve actuator rod 14 is actuated by coil winding 18, and is in fluid communication with a capillary outlet port 23. Normally, the inlet valve is under pressure from a supply pressure head and the outlet valve is under static loading of fluid trapped in the fluid chamber 26. The supply pressure head could be as simple as a reservoir at a sufficient head.

All three magnetostrictive actuator rods 12, 14, 16 are formed of magnetostrictive material such as Terbium alloy, Dysprosium, and Iron that is commercially available in the form of rods, tubes, washers, and particles. For example, all of the magnetostrictive rods 12, 14, 16 may be formed of Terfenol-D particles that are 50 to 300 microns in size, distributed uniformly in a polymeric host material. These particles are preferably magnetically oriented in a desired direction by applying an orientation magnetic field during fabrication. This results in a composite Terfenol-D rod, the crystalline structure of which is such that all the magnetic domains produce maximum moments when excited in the preferred direction. Such magnetostrictive components may be made in bulk form or can be made of magnetostrictive particulate composite. Although the magnetostrictive particulate composite rods are less efficient compared to the bulk rods, they can be shaped to almost any shape. Also, the magnetostrictive particulate composite rods have higher bandwidth. The magnetostrictive rods will elongate by up to 2000 parts per million, and the frequency response of rods can be as high as 10 kHz. The magnetostrictive piston actuator rod 12 is preferably about 2 mm diameter, and is preferably biased with a permanent magnet to facilitate bi-directional actuation. Rods 14 and 16 may be the same size or 1 mm or smaller magnetostrictive rods. The rods may also contain an internal bore so that a preload stress can be applied to the actuator.

In operation, each of the magnetostrictive elements are operated in a cycle comprising a fluid induction step and fluid dispensing step under the following preferred conditions:

Step 1: Fluid Induction

Piston Actuator Rod 12 is normally extended with no field using a bias permanent magnet;

Inlet valve actuator rod 16 is retracted with no field,

Outlet valve actuator rod 14 is extended using a bias permanent magnet with no field

Thus, the fluid dispensing sequence is explained as follows:

Simultaneously and synchronously, the inlet valve actuator coil 20 is not excited, the inlet valve actuator rod 16 is retracted, and the capillary inlet port 22 remains open. The outlet valve actuator rod 14 is normally extended and closed while the inlet valve actuator rod 16 is normally open. The piston element coil 10 is excited, whereby piston actuator rod 12 retracts causing fluid to be sucked into the fluid chamber 26 via inlet port 22. Fluid chamber 26 completely fills.

Fluid Dispensing:

The next step of each discrete pump cycle is the fluid dispensing step. First the inlet valve actuator rod 16 is actuated to close the inlet port 22. The outlet valve actuation coil 18 is excited to retract the outlet valve actuator rod 14, thereby opening the capillary outlet passage 23. Next, the coil winding 10 is deactivated, thereby extending the piston actuator rod 12, causing the piston actuator rod 12 to positively displace fluid from fluid chamber 26 (pushing the fluid into the capillary outlet 23.

The cycle then repeats as described above, as the outlet valve actuation coil 18 is deenergized causing the outlet valve to be closed and moving more fluid into capillary 23. The inlet valve actuation coil 20 is deenergized leaving the inlet valve in an open status.

The above-described pump cycle may be controlled by a micro-controller, conventional personal computer or a dedicated programmable logic controller (PLC). One skilled in the art will understand that any number of inlet and/or outlet valves may be provided by adding additional actuation coils, outlet valve actuators, and capillary passages, and by maintaining proper synchronization with programming of the control computer or PLC. Thus, multi-outlet port fluid dispensing is possible for various filling and dispensing applications.

The above-described magnetostrictive pump with gated fluid inlet and outlet channels is especially well-suited for use as a cell sorter with magnetostrictive gating mechanism. Cell sorters sort cells based on desired characteristics, usually measured optically.

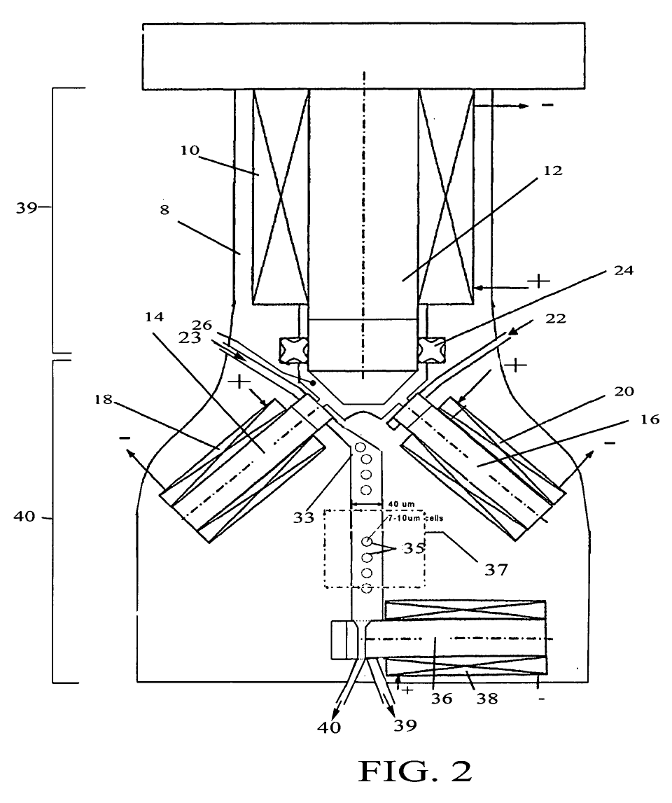

FIG. 2 illustrates the pump of FIG. 1 adapted to work as a cell sorter gating system. Here the pump housing 8 defines a pumping section 39 and a capillary detection and sorting section 40. As above, the pump actuator rod 12 enters the fluid chamber 26 from its own chamber, rod 12 being sealed by an O-ring seal 24 that encircles the lower distal end. The pump actuator rod 12 should be capable of making precise micro-displacements between the fully opened and fully closed two fixed stops. The cell-containing fluid is generally moved incrementally down the capillary 33 to a detection window 37 in either the capillary 33. The small volume defined in the detection window 37 corresponds to the size of the fluid being pumped incrementally in capillary 33. The capillary can be 25-100 microns in diameter and depends on the material being sorted. The motion of the positive displacement piston rod 12 combined with the piston area defines the incremental volume for each pump cycle. Each positive displacement rod 12 movement is adjusted between two fixed stops, fully open and fully closed. The lengthwise motion of magnetostrictive rods for high speed is usually less than 50 microns. Magnetostrictive actuator 36 is normally retracted with the incremental fluid passing through the valve into detection window 37 via inlet 33. A gate valve comprised of another magnetostrictive gating actuator 36 and excitation coils 38 is located below the detection window 37 by an amount corresponding to the incremental fluid volumes being dispensed. The magnetostrictive gating actuator 36 is formed with an axially-positionable passthrough capillary that can be selectively positioned to dispense cells through any of a plurality of capillary output ports (two are shown). The entire fluid system is driven by incremental motion defined by piston 12 and is capable of dispensing on digital command. The incremental fluid motion can be stopped or can be run at any predefined speed up to the maximum speed defined by the magnetostrictive rods. The detection window 37 is used to optically investigate cell volumes for sorting. If the detection system defines a cell or group of cells to be sorted the incremental volume is completely removed when it reaches the gating valve (gating actuator 36). The gating actuator 36 is energized by coils 38 to move the passthrough capillary therein to align it with the proper output capillary 39, 40.

In operation, a pumping sequence begins with a cell induction step followed by a pumping step as defined earlier with regard to FIG. 1. As the fluid enters the detection window 37 the pump is stopped and an optical investigation takes place. For stem cell sorting a fluorescent and light scatter analysis is performed on each fluid volume. It the detection system identifies a fluid volume to be collected it is removed from the normal fluid stream by actuation of valve 36 to position alignment to output capillary 39. Using an incremental digital pumping system fluid volumes can be completely stopped if further analysis of the optical signal is needed. This capability allows the system to use a rapid optical read to be made and if an event of interest is in the detection window further optical analysis can be made before moving the sample out of the digital analysis window.

In addition to sorting fluorescent and non-fluorescent cells, the invention can also provide multiparameter analysis, such as multicolor detection. For example, cells labeled with different color markers or dyes, or cells labeled with other detectable reagents can be sorted by the invention.

One skilled in the art will understand the choice of normally-open versus normally closed positions of the actuator rods 14, 16 and 36 are a matter of design choice. In regard to primary actuator rod 12, it should be capable of moving around 1000 parts per million or around 1 micron per millimeter. For a movement of 25-30 microns, a rod of this type would need to be around 25-30 mm long. Any rod of any material that can expand is within the scope of the present invention. The gating rods, including each of the magnetostrictive actuator rods 14, 16 and 36 as shown in FIG. 2, should be capable of operating at speeds up to 10,000 Hz. The above-described cell sorter system couples the action of a precision pump, an optical detection system and a sorting gate mechanism in one small unit.

In the foregoing embodiments the motion of the positive displacement piston rod 12 under PLC control (as well as all gate rods 14, 16, 36) was presumed to be between two fixed stops, fully open and fully closed. The position of the two stops may be determined quantitatively. However, it is possible to introduce an element of feedback into the positive displacement piston rod 12 and/or to any of the gating rods 14, 16, 36 to provide for qualitative start and stop positions.

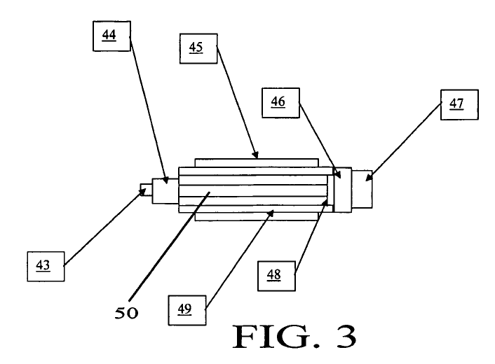

FIG. 3 shows an alternative embodiment of a magnetostrictive actuator with position-sensing feedback. The actuator has an actuator coil 49 and a sensing coil 45 about a magnetostrictive rod 50. When the magnetostrictive rod 50 is in it fully retracted normal position it is in a prestressed state as defined by the compressive forces generated by an annular collet tip holder 44 and a Belleville washer 46 and nut 47. When the magnetostrictive rod 50 returns to its unactivated position the normally retracted position will result in a stress induced signal that is sensed by the sensing coil 45. When the magnetostrictive rod 50 is extended to an open fixed stop again, a stress is induced resulting in a signal detected by the sensing coil 45. These sensing coil signals may be digitized in a known manner and sent to the PLC to allow the detection of position for each magnetostrictive rod in the systems described above. The use of a permanent magnetic material allows the magnetostrictive rod 50 to have an extension bias that can be removed by the actuator coil 49. This use of a bias magnet allows the rod 50 to be used in either a normally extended or retracted condition. Preferably, the magnetostrictive rod 50 bears a disposable tip 43 that is held in place by collet tip holder 44. The tips 43 may be made from stainless steel, ceramic or a polymer material capable of being in the system fluid flow. The above-described magnetostrictive actuator with position-sensing feedback may be substituted for any of the magnetostrictive actuators previously described to add the position-sensing feedback and digital control capability.

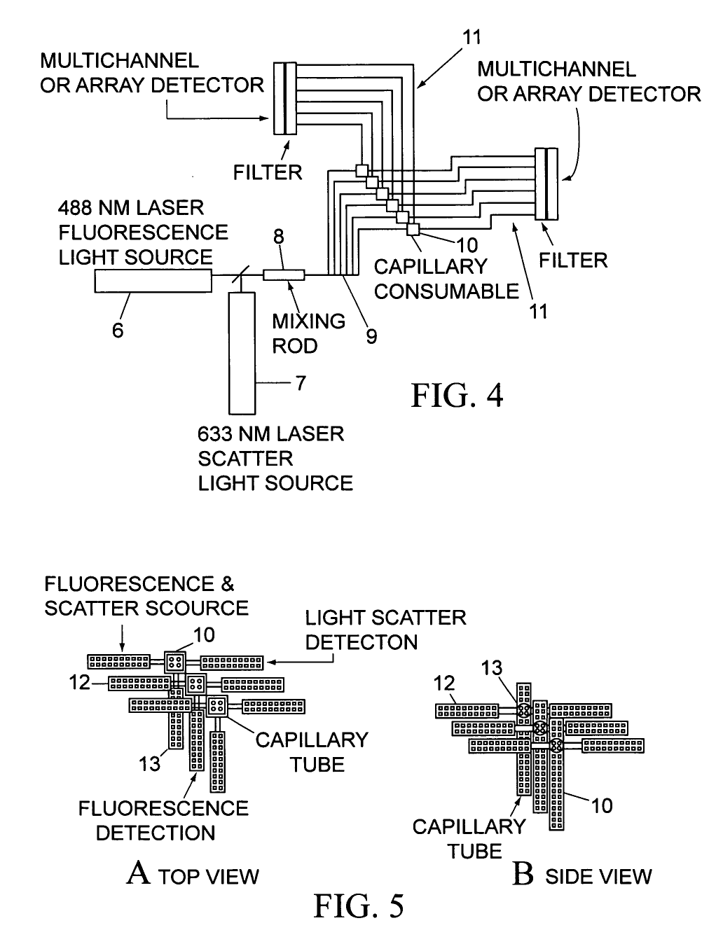

FIG. 4 defines an optical detection system that is suitable for use with the cell sorter embodiment of the present invention and interfaces to the system's detection window. For example, an argon 488 nm laser 6 acts as a fluorescence source while a helium neon 633 nm laser 7 acts as a light scatter source. The two lasers are coupled into a mixing rod 8 that is used to combine the source wavelengths and effectively couple light into optical light fibers 9. The light fibers 9 are used to bring light into capillary cells 10 that correspond to the optical detection windows. Pairs of other fibers 11 are used to couple fluorescence and light scatter from each capillary system into multi-channel array detectors 12. The multi-channel array detectors 12 can contain photo-multipliers or diode arrays or any other light detection means. Any other type of optical system or combination of wavelengths is within the scope of the present invention. The fiber optics 11 can be positioned as shown in FIG. 4 to allow for multiple cell sorter systems to be used with a multiple fiber optic array. Each capillary is positioned so that the optics for the source and detectors can be 90 and 180 degrees apart. Fluorescence detection can have its fibers one above another as shown in FIG. 4. The capillary detection modules can be round or square and can use optical refractive index matching fluids for coupling into the output fibers (or lens/fibers).

FIG. 5 shows the capillary tubes 10 from a top view (FIG. 5A) and a side view (FIG. 5B). A fluorescence and light scatter source 12 feeds the light of two different wavelengths into the capillaries 10. Fluorescence detection 13 and light scatter detection 14 can be positioned 90 degrees apart as shown in FIGS. 5A and 5B. The embodiments shown in FIGS. 5A and 5B are representative of the types that can be used with the present invention. The scope of the present invention includes many other embodiments of optical detection.

When either a fluorescence or light scatter signal (or both) indicates a cell to be sorted is in position, the pump control electronics keeps track of the time shift necessary so that the cells are properly presented to the gating mechanism (gating actuator 36 of FIG. 2). The gating mechanism can be exercised when the selected cell is in exactly the correct position. Both ports under the gating mechanism can have a vacuum source coupled to them. The opening of the proper port causes the selected cell to exit (be removed) through the correct output channel 39, 40. For example, one output channel 39 can be for selected cells and the other 40 for all else. Multiple channels (more than two) are within the scope of the present invention and can lead to more sophisticated sorting based on various cell properties.

When a selected cell is in position in the capillary 33, the control electronics either applies the external magnetic field via coils 38, or not, depending upon which exit port 39, 40 is desired. Once the magnetic field is removed (if it had been applied), the gating actuator 36 re-aligns itself to the normal position or non-selected normal port. The speed of fluid flow from the magnetostrictive pump can be high as previously discussed. When a cell needs to be removed the incremental flow can be stopped to be carefully synchronized to the gating valve (gating actuator 36).

The present invention thus couples a precision pump, an optical cell detection system and a control channel into a closed system that can move a cell into position to be identified, identify it and make a sort decision, move the selected cell to the control gate (if not already in position), set the control gate, and pull the selected cell out into a proper exit port. The present invention can be run in a pulsed (or discrete motion) mode or it can be run with continuous flow and hence continuous cell motion. Cells are identified and classified by the optical detection system according to optical properties that are either intrinsic or can be given to the desired cells through methods well-known in the art.

The present invention allows cells to be sorted by using a dynamic gate that can be constructed using magnetostrictive or other technology to create a small capillary valve. The valve can be switched from one state to another by the application of a magnetic field. The valve can be constructed where two (or more) ports are very close together so that as one port closes, the other is opening, or it can be constructed with ports further apart so that there is a period of time when both ports are blocked. The valve system can be part of a single or multiple capillary block. Such a block can be micro-machined using laser technology.

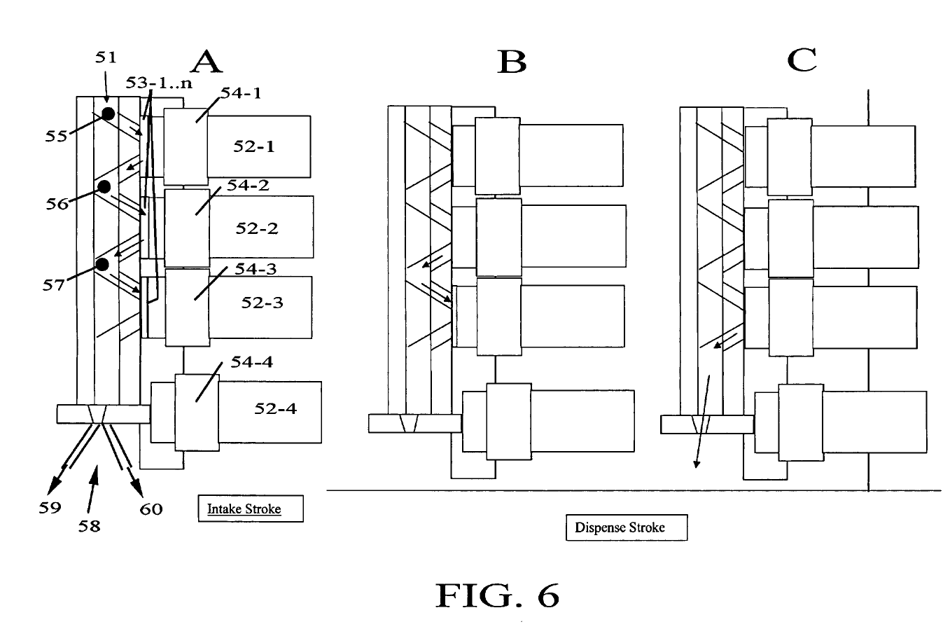

For example, FIG. 6 illustrates a disposable micro-machined microfluidic chip that can be manufactured via laser technology, and is illustrative of the sequence of operation including intake stroke and dispense stroke. The inlet 51 to the disposable microfludic chip is shown at the top of the device. The microfluidics chip can be made from several layers in order to simplify the connection of fluid ports. The inlet 51, positive displacement pistons 52-1 . . . 3, gate 52-4 and corresponding outlet ports (shown by arrows) operate as earlier defined. Each of the magnetostrictive actuators 52-1 . . . 4 is positioned on one side of the device allowing the microfluidics chip and valve tips to be discarded after each use. Like collets 54-1 . . . 4 are used to connect the magnetostrictive rods to the device. The fiber optic window for the source is shown as position 55. Ninety degree fluorescence is available in window 56 and forward light scatter in window 57. The optical analysis defines if the cell(s) or fluid in the detection window needs to be removed from the normal flow. Gating mechanism 58 and gate actuator 54-4 (as in gating actuator 36 as in FIG. 2). will position either to the normal flow 59 or selection 60 channel. A vacuum assist may be connected to channels 59 and 60 as a matter of deign choice and are within the scope of the present invention.

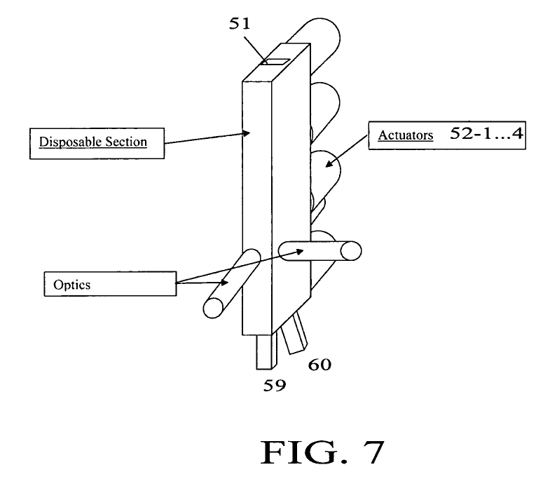

FIG. 7 is an overall perspective view of the disposable cell sorter micro-fluidic chip schematicized in FIG. 6, placed in the context of an overall sorting system. The loading of the disposable chip into the system would be to first connect all the actuator tips to the magnetostrictive rods using the collets. The inlet port connector 51 and outlet ports 59, 60 for selection and normal are then docked to the disposable device. The last connection is for the fiber optic source and detectors to the microchip. Once the system has been used the microchip is removed and discarded.

It should now be apparent that the above-described positive displacement pump design by virtue of its combination magnetostrictive fluid pump with magnetostrictive gates offers a high-throughput and high-accuracy filling and dispensing solution for various applications, with particular utility as a cell sorter.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

We claim:

1. A pump comprising:
a pump housing defining an internal fluid chamber, an inlet port in fluid communication with said internal fluid chamber, an outlet port in fluid communication with said internal fluid chamber, and at least three cavities in said pump housing, a first one of said cylindrical cavities being in fluid communication with said fluid chamber at one end, a second one of said cylindrical cavities being in fluid communication with said inlet port at one end, and a third one of said cylindrical cavities being in fluid communication with said outlet port at one end;
a pump actuator including a first actuator rod formed of magnetostrictive material seated in said first cavity, a coil winding about said first actuator rod for electrical excitation to produce a magnetic field for varying a length of said first actuator rod, said first actuator rod having an end protruding into said pump housing internal fluid chamber, and a fluid seal for sealing the coil winding from the protruding end of said pump housing internal fluid chamber;
an inlet port gate actuator including a second actuator rod formed of magnetostrictive material seated in said second cavity, a coil winding about said second actuator rod for electrical excitation to produce a magnetic field for varying a length of said second actuator rod, said second actuator rod having an end protruding into said inlet port within the pump housing; and
an outlet port gate actuator including a third actuator rod formed of magnetostrictive material seated in said third cavity, a coil winding about said third actuator rod for electrical excitation to produce a magnetic field for varying a length of said third actuator rod, said third actuator rod having an end protruding into said outlet port within the pump housing;
whereby said pump employs a pumping sequence including a fluid induction step wherein said first rod is retracted to induct fluid into said internal fluid chamber, and a fluid dispensing step wherein said first actuator rod is extended to dispense fluid through the outlet port.

2. The pump of claim 1, wherein fluid in said internal fluid chamber is displaced by a lengthwise extension of said first actuator rod.

3. The pump of claim 1, wherein said inlet port and said outlet port are formed as capillary channels leading from said internal fluid chamber to outside said pump housing.

4. The pump of claim 3, wherein said inlet port, outlet port and fluid chamber are fluidly coupled.

5. The pump of claim 1, wherein all of said first through third channels are cylindrical.

6. A pump comprising:
a pump housing defining an internal fluid chamber, an inlet port in fluid communication with said internal fluid chamber, an outlet port in fluid communication with said internal fluid chamber;
a pump actuator including a first actuator rod formed of magnetostrictive material seated in said pump housing, a coil winding about said first actuator rod for electrical excitation to produce a magnetic field for varying a length of said first actuator rod, said first actuator rod having an end protruding into said pump housing internal fluid chamber;
an inlet port gate actuator including a second actuator rod formed of magnetostrictive material seated in said pump housing, a coil winding about said second actuator rod for electrical excitation to produce a magnetic field for varying a length of said second actuator rod, said second actuator rod having a normally retracted end that can be selectively extended to protrude into and close said inlet port within the pump housing; and
an outlet port gate actuator including a third actuator rod formed of magnetostrictive material seated in said pump housing, a coil winding about said third actuator rod for electrical excitation to produce a magnetic field for varying a length of said third actuator rod, said third actuator rod having a normally extended end that protrudes into and closes said outlet port within the pump housing but can be selectively retracted to open said outlet port;
whereby said pump employs a pumping sequence including a fluid induction step wherein said first rods is retracted to induct fluid into said internal fluid chamber through said open inlet port, and a fluid dispensing step wherein said first and second actuator rods are extended and said third actuator rod is retracted to dispense fluid through the open outlet port.

7. A pump comprising:
a pump housing;
at least one actuator seated in said pump housing and including an actuator rod formed of magnetostrictive material, a first coil winding about said first actuator rod for electrical excitation to produce a magnetic field for varying a length of said actuator rod, and a second coil winding about said first actuator rod for sensing a stop position of said actuator rod.

8. The pump of claim 7, wherein said pump housing is defined by an inlet port, outlet port and fluid chamber all in fluid communication.

9. A cell sorter comprising:
a housing defining an internal detection chamber, an inlet port in fluid communication with said internal detection chamber, and at least two outlet ports in fluid communication with said internal detection chamber;
a detection system for determining a physical characteristic of cells in said detection chamber;
a pump actuator including a first actuator rod formed of magnetostrictive material protruding into said pump housing detection chamber, a coil winding about said first actuator rod for electrical excitation to produce a magnetic field for varying a length of said first actuator rod, and a fluid seal for sealing the coil winding from the internal detection chamber;
an inlet port gate actuator including a second actuator rod formed of magnetostrictive material, and a coil winding about said second actuator rod for electrical excitation to produce a magnetic field for varying a length of said second actuator rod; and
at least two outlet port gate actuators including a third actuator rod and fourth actuator rod both formed of magnetostrictive material, and coil windings about said third and fourth actuator rods for electrical excitation to produce a magnetic field for varying a length of said third and fourth actuator rods.

10. The cell sorter of claim 9 wherein said cell detection system is optical.

11. The cell sorter of claim 9 wherein said cell detection system uses fluorescence.

12. The cell sorter of claim 9 wherein said cell detection system uses scattered light.

13. The cell sorter of claim 12 wherein said cell detection system uses both fluorescence and scattered light.

14. The cell sorter of claim 9 wherein a fluorescence and scattered light determination is made simultaneously.

15. A cell sorter for sorting individual cells from undesired matter, comprising a precision magnetostrictive pump for pumping cell-containing fluid into a capillary and controlling positions of said cells in said capillary; an optical detection system for determining when a desired cell is in a predetermined position in said capillary; and a pair of capillary outlets each controlled by a magnetostrictive gate controlled by a magnetic field that causes a desired cell to pass through a cell exit port and waste material to pass through a waste port.

16. A pump, comprising:
a pump housing defining an internal fluid chamber, an inlet port to the fluid chamber, and an outlet port from the fluid chamber;
a positive displacement pump in communication with said fluid chamber and comprising a rod formed of magnetostrictive material and a coil windings for electrical excitation to produce a magnetic field that varies the length of the rod;
a pair of valves selectably blocking said inlet port to the fluid chamber and the outlet port from the fluid chamber, each of said valves comprising a rod formed of magnetostrictive material and a coil winding for electrical excitation to produce a magnetic field that varies the length of the rod;
a controller for controlling application of control signals to said coil windings to maintain a pumping sequence including a fluid induction step wherein the pump and inlet port valve rods are retracted and the outlet valve rod is extended to open the inlet port and induct fluid into the fluid chamber, and a fluid dispensing step wherein the pump and inlet port valve rods are extended and the outlet valve rod is retracted to close the inlet port and expel fluid from the fluid chamber.

17. The pump of claim 16, wherein said inlet port and said outlet port are formed as capillary channels leading from said internal fluid chamber to outside said pump housing.

18. The pump of claim 17, wherein said inlet port, outlet port and fluid chamber are fluidly coupled.
